(19) United States
(12) Patent Application Publication
(10) Pub. No.: US 2026/0009775 A1
(43) Pub. Date: Jan. 8, 2026

(54) AGRICULTURAL MACHINE WITH SWATH SENSORS

(71) Applicant: CLAAS Saulgau GmbH, Bad Saulgau (DE)

(72) Inventors: Josef Fischer, Biberach (DE); Michael Becker, Fronhausen (DE); Dennis Neitemeier, Lippetal (DE)

(73) Assignee: CLAAS Saulgau GmbH, Bad Saulgau (DE)

(21) Appl. No.: 19/259,435

(22) Filed: Jul. 3, 2025

(30) Foreign Application Priority Data
Jul. 3, 2024 (DE) 10 2024 118 936.3

(51) Int. Cl. G01N 33/00; A01D 78/00; G01N 21/3563; G01N 21/359; G01N 22/00

(52) U.S. Cl. CPC G01N 33/0098; A01D 78/001; G01N 21/3563; G01N 21/359; G01N 22/00

(57) ABSTRACT

An agricultural machine comprising a swath sensor system. The swath sensor system is configured to detect one or more properties of a swath on an agricultural area traversed by the agricultural machine. The agricultural machine further comprises a swather for swath generation. At least one sensor of the swath sensor system is configured to detect properties of a stream of swath-forming material during or after its processing into a swath by the swather.

### CROSS REFERENCE TO RELATED APPLICATION

This application claims priority under 35 U.S.C. § 119 to German Patent Application No. DE 10 2024 118 936.3 filed Jul. 3, 2024, the entire disclosure of which is hereby incorporated by reference herein.

### TECHNICAL FIELD

The present invention relates to an agricultural machine which is self-propelled or movable with the aid of a prime mover and which has a swath sensor system for detecting one or more properties of a harvested material swath.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

EP 3 365 649 B1 discloses an agricultural machine, such as a tractor, for pulling a baler behind it. A swath sensor system may comprise a radar sensor mounted on the front part of the tractor, for example on a hood or the front edge of a cab roof, in order to detect properties of a swath immediately before it is picked up by the baler.

The properties detected by the swath sensor system may include the moisture of the swath. By using the measured moisture, it may be decided whether or not the material of the swath is suitable for a planned use.

### BRIEF DESCRIPTION OF THE DRAWINGS

The present application is further described in the detailed description which follows, in reference to the noted drawings by way of non-limiting examples of exemplary embodiment, in which like reference numerals represent similar parts throughout the several views of the drawings, and wherein:

FIG. 1 illustrates a top view of an agricultural machine according to a first embodiment.

FIG. 2 illustrates a view of an agricultural machine according to a second embodiment.

FIG. 3 illustrates the sensor bar with the sensors distributed thereon.

### DETAILED DESCRIPTION

As discussed in the background, the measured moisture may be used to decide whether or not the material of the swath is suitable for a planned use. However, if it turns out that the material is too moist or too dry for the planned use, then at that point, time and energy has already been spent in moving the agricultural machine to the field, and this effort may be wasted if the planned processing cannot be performed.

Another property that may be monitored with the swath sensor system is the presence of foreign objects or bodies in the swath. For example, metallic foreign objects or bodies should not be picked up with the swath since they may lead to injury if these metallic foreign bodies get into the livestock feed. To avoid this, the tractor must be stopped in good time upon detection of a foreign object so that the foreign object may be removed. Abrupt braking is not only unpleasant for the driver; it may also damage the ground in that it leads to displacement of the soil and unevenness. With more gentle braking, the removal of the foreign body may become more difficult by the fact that the tractor is positioned above it.

By measuring the geometric properties of the swath, the amount of material contained in the swath may be estimated, and the transportation capacity required for its removal may be estimated, but only after the processing of the swath by the machine has already begun.

Thus, in one or some embodiments, to overcome at least one of the above-mentioned disadvantages, an agricultural machine with a swath sensor system is disclosed. The swath sensor system is configured to detect one or more properties of a swath on an agricultural area traversed by the agricultural machine. The agricultural machine further comprises a device for swath generation (e.g., a swather). Moreover, at least one sensor of the swath sensor system is arranged or positioned so as to be configured to detect the one or more properties of a stream of swath-forming material during and/or after its processing by the device (e.g., the swather).

By being provided on the swath-forming machine itself, the swath sensor system may therefore enable early detection of one, some, or all properties of interest.

If, for example, at the time of swath formation, its moisture is already measured, its further development may be predicted quite accurately based on the weather conditions, and the desired further processing may be performed at a designated time, such as the earliest possible time at which the residual moisture content of the swath permits this.

Further, a foreign body already detected at the time of swath formation may be removed at any suitable point before further processing of the swath; even if the foreign body is not removed by then, by knowing the location of the foreign body, a machine performing further processing may be stopped in sufficient time before the foreign body is reached without the need for abrupt braking.

Based on information about the amount of material contained in the swath already obtained at the time of swath generation, sufficient transport capacity may be provided to remove the material without having to improvise at short notice.

Thus, in one or some embodiments, at least one sensor of the sensor system may be aligned to a region of the agricultural area behind the device (e.g., the swather) in the direction of travel of the agricultural machine in order to detect the one or more properties of a swath generated by the device.

In particular, a non-contact sensor such as any one, any combination, or all of a camera, a radar sensor, a lidar sensor or a near infrared (NIR) sensor may comprise such a sensor. Other non-contact sensor types may be considered for detecting the one or more properties of a stream of the swath-forming material before the formation of the swath. For example, if the device for swath creation is a rotary swather, a hub sensor that is arranged or positioned in a hub of the rotor may be used a sensor in order to detect a torque that material set in motion by the rotor during swathing exerts on the rotor, or a swath guard sensor that responds to material impacting a swath guard.

The sensor system may be configured to supply or generate measurement data of one or more properties, each linked to a location of their measurement (e.g., each correlated to a corresponding location of their respective measurement). Based on this data, a location at which, for example, a foreign object was detected in the swath at the time of swath formation may be found again at a later time and the foreign object removed, or the location at which the loading capacity of a collection machine may be exhausted during subsequent collecting of the swath and transferring from the collection machine to a transport vehicle is necessary may be precisely predicted, and rapid collection may be performed by providing the transport vehicle as required.

The one or more properties may relate to any one, any combination, or all of: swath height; swath width; swath cross-section; position of a swath top and/or bottom; dry matter and/or water content; swath density; swath homogeneity; and contained plant species. Measured values on the dimensions of the swath and its composition may be merged to obtain an estimate of the amount of material contained in the swath. Data on density or homogeneity may allow for the identification of foreign bodies, clumped, matted or twisted material that may cause difficulties in the collection of the swath or its subsequent processing.

In order to precisely detect the condition of the swath, the swath sensor system may comprise a plurality of sensors offset against each other transverse to the direction of travel. These plurality of sensors may each detect the same measured variable, such as a height or layer thickness, at different locations of the swath cross-section. However, in one or some embodiments, it is also contemplated to arrange a plurality of spatially-resolving sensors of different types in such a way that their detection regions only partially overlap; since the sensor system learns data from both sensors obtained in the overlapping region since these are normally combined, the sensor system may estimate the missing data for a region outside the overlapping area for which data from one of the sensors is missing, using data measured there from at least one other sensor.

Furthermore, at least one sensor may be arranged or positioned to detect at least one property of parts of the surface laterally adjacent to the swath. Such data, for example regarding surface condition and/or surface moisture, may be helpful in predicting the development of the moisture content of the swath and in selecting a suitable time for collection. However, they may also serve to control the swath creation, for example by detecting the density of material suitable for forming the swath which has not been detected by the device for swath creation and which has therefore remained to the side of the swath, and using the measured values obtained in this way to control the swath-creating device.

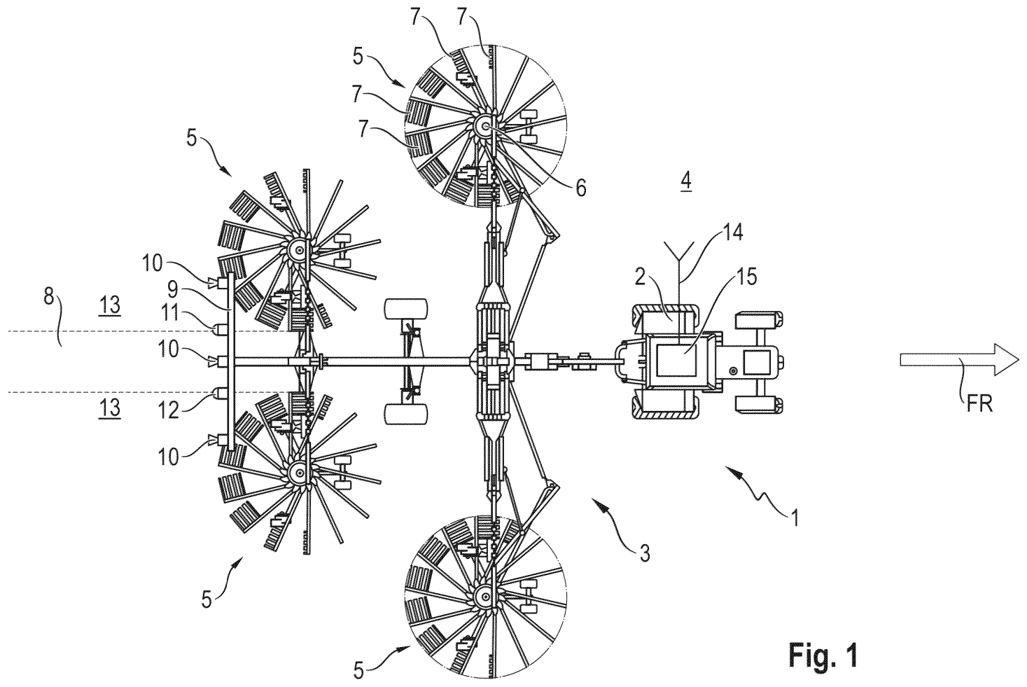

Referring to the figures, FIG. 1 shows a top view of a combination 1 comprising (or consisting of) a tractor 2 and a swather 3 pulled by the tractor 2 on an agricultural area 4. Swathers are disclosed in US Patent Application Publication No. 20140202566 A1; U.S. Pat. Nos. 11,917,943; 12,089,533; and US Patent Application Publication No. 2025/0036148 A1, each of which are incorporated by reference herein in their entirety. A swather, alternatively termed a windrower, is a farm implement that may cut hay or small grain crops and may form them into a windrow for drying. Generally speaking, the swather may be self-propelled with an engine, or drawn by a tractor and power take-off powered.

The swather 3 may comprise a plurality of pairs of rotors 5, such as two (as depicted), following one another in the direction of travel FR of the combination. The rotors 5 may be driven in rotation (in a manner known per se via a power take-off shaft of the tractor 2) and have one or more rakes 7 projecting radially from a hub 6. By pivoting the rakes 7 against the ground during part of their rotation around the hub 6, the rakes 7 may pick up or collect plant material lying there and throw it towards the center of the swather 3 so that a swath 8 is formed there which remains behind the combination 1 on the area 4.

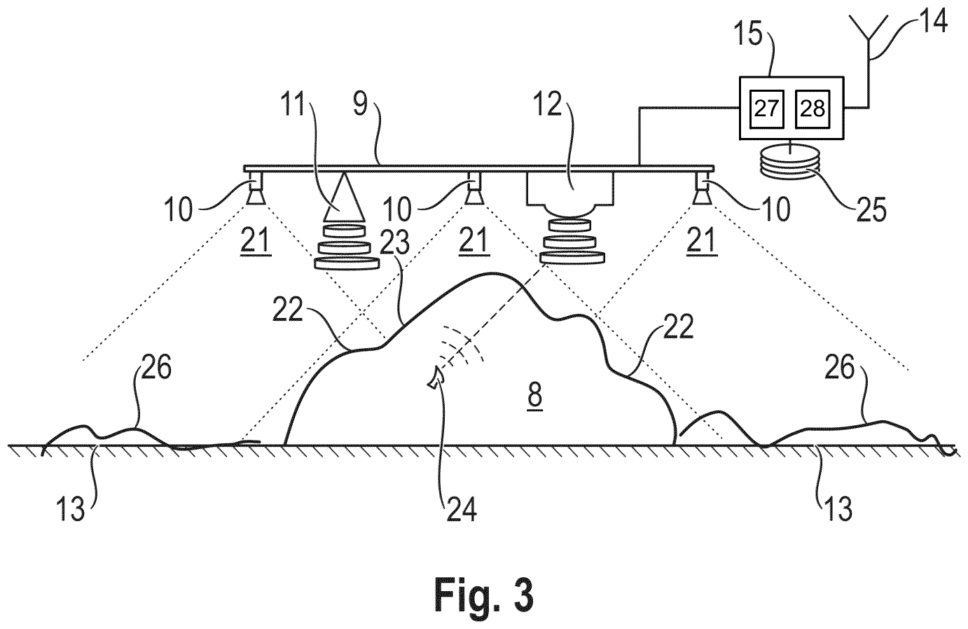

A sensor bar 9 may be mounted at the rear of the swather 3 transverse to the direction of travel. The sensor bar 9 may carry a plurality of sensors 10, 11, 12, which may be aligned with the swath 8 and regions 13 of the area 4 on both sides of the swath 8. The sensors 10, 11, 12 are described in more detail with reference to FIG. 3.

Other sensors may be used. As one example, hub sensors, of known design, may be included on each hub 6 in order to detect the torque acting between the drive and the rotor 5.

In one or some embodiments, the sensors 10, 11, 12 and a satellite navigation system 14 may communicate (e.g., wired and/or wirelessly) with a computer 15 on board the tractor 2. In one or some embodiments, the computer 15 may be permanently installed in the tractor 2;

alternatively, the computer 15 may comprise a mobile computer (e.g., a notepad or smartphone), which is carried by a driver of the tractor 2.

In one or some embodiments, the computer 15 may comprise at least one processor, which may work with a memory, such as mass memory 25 and/or another type of memory. The memory, such as mass memory 25, may comprise at least one memory configured to store data, such as sensor data, and/or computer-executable instructions stored on the tangible memory. Moreover, at least one communication interface 28 (configured to communication with devices external to the computer 15, such as sensor 10, 11, 12, other electronic devices, or the like). The at least one processor and at least one memory may be in communication (e.g., wired and/or wirelessly) with one another. In one or some embodiments, the computer 15 may comprise a microprocessor, controller, PLA, or the like. Similarly, the mass memory 25 may comprise any type of storage device (e.g., any type of memory, such as RAM, ROM, or a combination thereof). Though the computer 15 and the mass memory 25 are depicted as separate elements, they may be part of a single machine, which includes a microprocessor (or other type of controller) and a memory. Alternatively, the computer 15 may rely on the mass memory 25 for all of its memory needs. Still alternatively, the computer 15 may rely on a database for some or all of its memory needs. The mass memory 25 may comprise a tangible computer-readable medium that include software that, when executed by the computer 15 is configured to perform any one, any combination, or all of the functionality described herein.

The computer 15 and the mass memory 25 are merely one example of a computational configuration for the electronic devices discussed herein. Other types of computational configurations are contemplated. For example, all or parts of the implementations may be circuitry that includes a type of processor, including an instruction processor, such as a Central Processing Unit (CPU), microcontroller, or a microprocessor; or as an Application Specific Integrated Circuit (ASIC), Programmable Logic Device (PLD), or Field Programmable Gate Array (FPGA); or as circuitry that includes discrete logic or other circuit components, including analog circuit components, digital circuit components or both; or any combination thereof. The circuitry may include discrete interconnected hardware components or may be combined on a single integrated circuit die, distributed among multiple integrated circuit dies, or implemented in a Multiple Chip Module (MCM) of multiple integrated circuit dies in a common package, as examples.

The satellite navigation system 14 may include a satellite receiver (such as a GPS receiver) in order to use satellites to provide location and timing information to the satellite receivers on the combination 1, such as the tractor 2 (e.g., the corresponding location when measurement data from sensor(s) is generated).

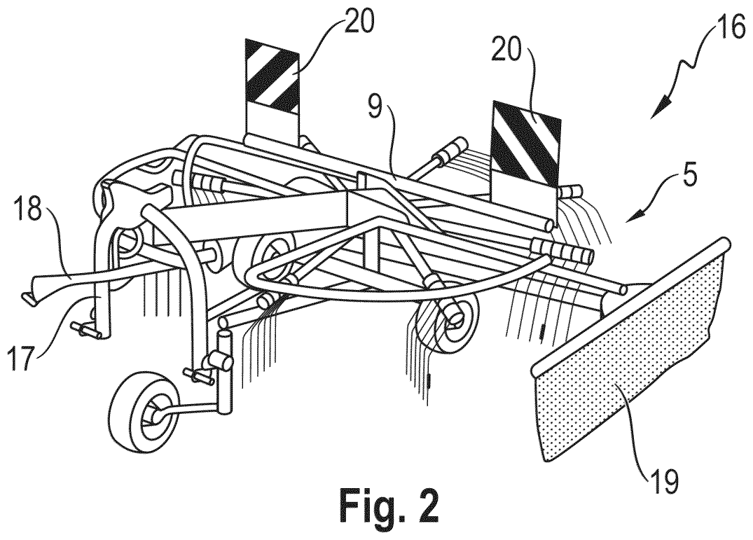

FIG. 2 illustrates a swather 16 with a single rotor 5 in an oblique view from the front, against the direction of travel. A yoke 17 for coupling to the tractor 2 and a shaft 18 for connecting to a PTO shaft of the tractor 2 may be seen at the front of the swather 16. Material accelerated sideways by the rotation of the rotor 5 strikes an elongated swath guard 19 here in the direction of travel and may form the swath (not shown in FIG. 2) by falling to the ground against the swath guard 19. In one or some embodiments, a sensor, configured to generate sensor data indicative of the amount of impacting material, may be integrated into the swath guard. Various sensors are contemplated. As one example, a capacitive sensor may be used as suitable for this purpose, such as described in the unpublished document DE 10 2024 113 247.7.

In one or some embodiments, a crossbeam, to which warning signs 20 for road traffic are conventionally attached, may serve as the sensor bar 9.

FIG. 3 illustrates the sensor bar 9 with the sensors 10, 11, 12 distributed thereon. In one or some embodiments, the sensors 10 may comprise cameras. The cameras 10 may be identical to each other or may differ in their spectral sensitivity. While most electronic cameras are provided with three sensor types for the colors red, green and blue, whose spectral sensitivity in each case simulates that of the sensory cells of the human eye, at least one of the cameras 10 may also comprise a different number of sensor types, including those sensitive to spectral ranges in the infrared or ultraviolet, or sensor types that are sensitive to a narrow-band of characteristic color tones of a specific component of the swath and whose measurement signal therefore allows a reliable inference of the presence or concentration of this component in the swath 8. In this regard, cameras of the same or different sensitivity are contemplated.

Solid angles 21, in which the cameras 10 are sensitive, are delimited by dotted lines in FIG. 3 (e.g., indicating respective areas of sensing). As shown in FIG. 3, the respective areas of sensing for at least two of the plurality of sensors overlap. In this regard, regions 22 (such as areas) of the swath 8 that lie in the overlapping area of the solid angles 21 of two cameras 10 may be measured stereoscopically. The distance between two cameras 10 may be greater than with most stereoscopic measuring systems; in particular, the distance may be greater than half the distance of the cameras 10 from the ground so that a region 23 of the top of the swath that lies outside the overlapping region of the solid angles 21 cannot be measured stereoscopically; however, the course, progression, or path of the top in this region 23 may be estimated by the computer 15 using measured values of the neighboring regions 22.

Alternatively or additionally, data from the lidar or radar sensors 11, 12 arranged or positioned between the cameras 10 may be used to estimate the course, progression, or path of the top of the swath in region 23. Since these sensors 11, 12 are based on a time-of-flight measurement, they may each provide measurement data on the top side contour on their own; unlike the cameras 10, data from a plurality of sensors does not have to be combined in order to be able to calculate the height of a point on the top side of the swath above the ground.

Furthermore, in one particular example, the radar sensor 12, since the radiation it emits penetrates the swath 8, may provide further information about its structure. The intensity of an echo reflected from the top side of region 23 and from inhomogeneities in the interior of the swath 8 may allow an inference to be made about the density of the swath 8; by using an echo from the ground surface, its height may be estimated, which may be important for calculating the swath cross-section and therefore ultimately the amount of material contained in the swath 8.

By using the radar echo it generates, the position of a foreign object 24 in the swath may be determined.

Color information supplied by the cameras 10, potentially supported by pattern recognition based on images from the cameras 10, may make it possible to identify any one, any combination, or all of the plant species contained in the swath 8, their proportion in the swath 8, and the degree of drying, which may also influence the calculation of the amount of material.

In one or some embodiments, an NIR sensor may be provided instead of (or in addition to) one of the sensors 11, 12 in order to obtain information about the properties of the swath, such as the composition by species and/or ingredients (e.g., the water or nutrient content). The NIR sensor may differ from an NIR-sensitive camera 10 in that the intensity of the IR radiation is not integrated over a broad spectral interval, but is detected with wavelength resolution, which may allow a much more precise inference about the chemical composition of the investigated material; however, spatial resolution may only be possible to a very limited extent by changing the spatial direction from which radiation is collected and analyzed, optionally with the aid of scanning optics.

While the sensor bar 9 is moved over the swath 8 freshly produced by the swather 3 or 16 during the operation of the combination 1, any one, any combination, or all of sensors 10, 11, 12, the hub sensors, the sensor of the swath guard 19, or the NIR sensor may collect data on successive slices of the swath 8 in the direction of travel FR. This data may not necessarily be suitable for calculating the material content therefrom of such a slice according to a deterministic formula; nevertheless, there are combinations of output data from the various sensors which, in their combination, may be related to certain values of properties of the slice such as composition, density, moisture content and the like and which, in their combination with each other, may allow an inference about the sought parameters. In one or some embodiments, the computer 15 may be programmed to save only the sensor data values supplied by the sensors in a mass memory 25 during use, in each case linked or correlated to the respective location, where the sensor data values were obtained, may be detected by the navigation system (e.g., via a GPS receiver on the tractor 2), so that an evaluation of the data collected in this way may take place at a later time, potentially by another, more powerful computer. If the computer 15 has sufficient computing power, it may also undertake an evaluation of the data during use and may also enter the results of the evaluation in the mass memory 25, together with the given locations of the measurements, potentially together with the data originally received from the sensors at the relevant location.

The evaluation may comprise (or consist of) identifying known combinations of values or those that are similar to known combinations in the detected sensor data and assigning the corresponding values of properties of a swath to the current swath 8 on the basis of previously determined values of properties of a swath corresponding to these combinations. For example, for each slice of the swath corresponding to a set of sensor data, the computer 15 may automatically estimate a mass of the slice or its space requirement on the vehicle used for collection; this data may, in turn, be used by the computer 15 (or another computing device) to automatically predict at which points along the path traveled by this vehicle on the field when collecting the swath 8 it may be necessary to transfer the collected material onto a transport vehicle, and to accordingly automatically coordinate the movements of the vehicles.

Responsive to an evaluation of the outputs of the sensors already performed by the computer 15 during use, the computer 15 may automatically control the agricultural machine (e.g., stop the combination 1) responsive to a foreign object 24 (e.g., a predetermined object in the swath-forming material) being detected in the swath 8 during the evaluation in order to give the driver the opportunity to inspect the foreign object 24 and, if necessary, remove it from the swath 8. The delay of the combination 1 required to stop may be kept to a minimum since, unlike in the prior art cited above, there is no risk of the foreign object 24 being picked up by a baler, for example, shortly after being detected and therefore becoming unreachable. Under certain circumstances, the foreign object 24 may also initially remain in the swath 8, because if the location of the foreign object 24 is still known at the start of a collection operation taking place at a later time, an agricultural vehicle that performs collection may be carefully stopped before reaching this location, knowing the position of the foreign body, and the driver may then be requested to remove the foreign body. It is also contemplated that the foreign object is not removed at all during collection, but that a material pick-up tool of the collecting vehicle is temporarily automatically raised in good time (e.g., at least a part of the swather, responsive to automatically detecting the foreign object, may be controlled, such as the respective rotor 5 may be automatically commanded to raise at least a predetermined height) before reaching the foreign object 24 so that the foreign object 24 remains on the field together with surrounding material of the swath 8. The material left behind with the foreign object 24 therein may then be removed at a later time, such as removed by an autonomous machine controlled to remove the foreign object 24. As discussed above, a respective location of the foreign object 24 (such as generated via a GPS receiver) may be used to effectively tag where the autonomous machine is to automatically travel in order to remove the foreign object 24.

The solid angles 21 of the two cameras 10 mounted at the ends of the sensor bar 9 may also detect the regions 13 of the area 4 on both sides of the swath 8 and any material 26 remaining thereon. The density of this material 26 in the regions 13 may be evaluated by the computer 15 in real time in order to be able to adjust the height of the rotors 5 of the swather 3 or 16 thereto. If this density lies above a limit value on at least one side of the swath 8, the computer 15 may automatically cause (e.g., by issuing one or more commands) the swather to lower the rotor or rotors 5 acting on this respective side by a predetermined increment in order to detect the material 26 more completely and transfer it into the swath. If the computer 15 thereafter determines that this lowering causes the limit value to be undershot, the height reached by the rotor 5 may be maintained. An abrupt increase in the torque detected by the hub sensor of the relevant rotor 5 indicates ground contact by the rotor 5 and causes the computer 15 to automatically raise the relevant rotor 5. In this regard, the computer 15 may automatically monitor the sensor data generated by the hub sensor (with torque an example of one aspect of monitoring operation of the swather) in order to determine whether there is an abrupt increase in the torque (e.g., the torque within a predetermined time period increases by at least a predetermined amount). Responsive to this determination of abrupt increase in the torque, the computer 15 may automatically command the swather 3 or 16 to automatically raise (e.g., by a predetermined amount) the height of the respective rotor 5.

Further, it is intended that the foregoing detailed description be understood as an illustration of selected forms that the invention may take and not as a definition of the invention. It is only the following claims, including all equivalents, that are intended to define the scope of the claimed invention. Further, it should be noted that any aspect of any of the preferred embodiments described herein may be used alone or in combination with one another. Finally, persons skilled in the art will readily recognize that in preferred implementation, some, or all of the steps in the disclosed method are performed using a computer so that the methodology is computer implemented. In such cases, the resulting physical properties model may be downloaded or saved to computer storage.

### LIST OF REFERENCE NUMBERS

1 Combination
2 Tractor
3 Swather
4 Agricultural area
5 Rotor
6 Hub
7 Rake
8 Swath
9 Sensor bar
10 Sensor/camera
11 Sensor/Lidar sensor
12 Sensor/radar sensor
13 Region (of area 4)
14 Satellite navigation system
15 Computer
16 Swather
17 Yoke
18 Shaft
19 Swath guard
20 Warning sign
21 Solid angle
22 Surface region
23 Surface region
24 Foreign body
25 Mass memory
26 Communication interface
27 Input/output device

1. An agricultural machine comprising:
a device for swath generation; and
a swath sensor system configured to detect one or more properties of a swath on an agricultural area traversed by the agricultural machine, wherein the swath sensor system comprises at least one sensor configured to detect one or more properties of a stream of swath-forming material at one or both of during or after its processing by the device for swath generation.

2. The agricultural machine of claim 1, wherein the at least one sensor of the swath sensor system is aligned with a region of area located behind the device for swath generation in a direction of travel of the agricultural machine in order to detect the one or more properties of the swath generated by the device for swath generation.

3. The agricultural machine of claim 1, wherein the at least one sensor comprises one or more of a camera, a radar sensor, a lidar sensor or a near infrared (NIR) sensor.

4. The agricultural machine of claim 1, wherein at least one sensor of the swath sensor system comprises a hub sensor or a swath guard sensor of a rotary swather.

5. The agricultural machine of claim 1, wherein the swath sensor system is configured to generate measurement data indicated of the one or more properties each correlated to a corresponding location of their respective measurement.

6. The agricultural machine of claim 1, wherein the one or more properties comprise one or more of: swath height; swath width; swath cross-section; position of a swath top or bottom; dry matter or water content; swath density; swath homogeneity; or contained plant species.

7. The agricultural machine of claim 1, wherein the one or more properties comprise three or more of: swath height; swath width; swath cross-section; position of a swath top or bottom; dry matter or water content; swath density; swath homogeneity; or contained plant species.

8. The agricultural machine of claim 1, wherein the one or more properties comprise a progression of a top of the swath-forming material.

9. The agricultural machine of claim 1, wherein the swath sensor system comprises a plurality of sensors offset relative to one another transverse to a direction of travel of the agricultural machine and including respective areas of sensing; and
wherein the respective areas of sensing for at least two of the plurality of sensors overlap.

10. The agricultural machine of claim 1, wherein the at least one sensor is positioned to detect the at one or more properties of parts of the agricultural area laterally adjacent to the swath.

11. The agricultural machine of claim 10, wherein the one or more properties comprise a concentration of swath-forming material on the parts of the agricultural area.

12. The agricultural machine of claim 1, wherein the swath sensor system comprises a plurality of sensors of different types; and
wherein the different types comprises one or both of: different spectral ranges; or different narrow-band characteristic color tones.

13. The agricultural machine of claim 1, further comprising at least one processor configured to:
determine, based on sensor data generated from the swath sensor system, a predetermined object in the swath-forming material; and
responsive to determining the predetermined object in the swath-forming material, automatically control operation of the agricultural machine.

14. The agricultural machine of claim 13, wherein the at least one processor is configured to automatically stop the agricultural machine responsive to determining the predetermined object in the swath-forming material.

15. The agricultural machine of claim 14, wherein the device for swath generation comprises at least one swather; and
wherein the at least one processor is configured to determine the predetermined object in a respective portion of swath-forming material and automatically stop the agricultural machine prior to the at least one swather generating the swath for the respective portion of swath-forming material.

16. The agricultural machine of claim 13, wherein the device for swath generation comprises at least one swather; and
wherein the at least one processor is configured to automatically raise to at least a predetermined height one or more rotors of the at least one swather.

17. The agricultural machine of claim 1, wherein the device for swath generation comprises at least one swather; and
further comprising at least one processor configured to:
determine, based on sensor data generated from the swath sensor system, at least one aspect of the swath-forming material or operation of the at least one swather; and
responsive to determining the at least one aspect of the swath-forming material or the operation of the at least one swather, automatically control operation of the at least one swather.

18. The agricultural machine of claim 17, wherein the at least one aspect of the swath-forming material comprises density; and
wherein the at least one processor is configured to adjust height one or more rotors of the swather based on the density of the swath-forming material.

19. The agricultural machine of claim 17, wherein the operation of the at least one swather comprises torque of a respective rotor of the at least one swather; and
wherein the at least one processor is configured to adjust height of the respective rotor based the torque of the respective rotor of the at least one swather.
